(19) United States
(12) Patent Application Publication
(10) Pub. No.: US 2026/0108639 A1
(43) Pub. Date: Apr. 23, 2026

(54) COMPLEX FOR DIAGNOSING NON-ALCOHOLIC STEATOHEPATITIS, USE, AND CONTRAST AGENT THEREOF

(71) Applicant: National Atomic Research Institute, Taoyuan (TW)

(72) Inventors: Hung-Wen YU, Taoyuan (TW); Mei-Hui WANG, Taoyuan (TW); Chung-Li HO, Taoyuan (TW); Kun-Liang LIN, Taoyuan (TW); Chen-Hsin CHAN, Taoyuan (TW); Wan-Chi LI, Taoyuan (TW)

(21) Appl. No.: 18/921,265

(22) Filed: Oct. 21, 2024

(51) Int. Cl. A61K 51/04

(52) U.S. Cl. CPC A61K 51/0491; A61K 51/0497

(57) ABSTRACT

Provided herein are a complex for diagnosing non-alcoholic steatohepatitis, use of the complex, and a contrast agent comprising the complex. The complex for diagnosing non-alcoholic steatohepatitis comprises hexa-lactoside, a metal chelator coupled to the hexa-lactoside, and a radionuclide labeling the metal chelator. The complex is capable of binding to an asialoglycoprotein receptor, so that the complex has potential for use as a radiological diagnostic agent.

### BACKGROUND

### Technical Field

The present invention relates to the field of radiological imaging and diagnosis, and in particular, to a complex capable of binding to an asialoglycoprotein receptor (ASGPR).

### Related Art

Non-alcoholic fatty liver disease (NAFLD) is a common chronic liver disease that may lead to non-alcoholic steatohepatitis (NASH) when too much fat accumulates in the liver. Further deterioration of NASH may lead to liver cell damage, liver fibrosis, or even liver cirrhosis or liver cancer. Therefore, early diagnosis of NASH is crucial for the prevention of liver diseases.

Currently, the gold standard for diagnosing NASH is a pathological section examination for liver tissue, examining specific pathological features. However, this diagnostic method is invasive and requires a liver puncture for sampling. Due to mild symptoms in the early stage of disease, it is usually difficult for patients to undergo the liver puncture, making it difficult for doctors to accurately diagnose the disease.

There are other non-invasive diagnostic methods as follows: (1) An abdominal ultrasound examination is carried out with a biochemical test for liver function indexes (such as GOT and GPT) to determine the presence of steatohepatitis. However, relying on liver function indexes alone cannot provide absolute confirmation of steatohepatitis, so this diagnostic method has limited accuracy. (2) Liver stiffness is measured by using a liver fibrosis scanner (Fibroscan) to assess the degree of liver fibrosis, but fibrosis is usually an advanced manifestation of NASH, so this cannot provide a reliable diagnosis in the early stage of disease.

In view of this, there is an urgent need in the art to provide a method for diagnosing non-alcoholic steatohepatitis early, accurately, and non-invasively, to overcome the defect in the related art.

### SUMMARY

For readers to understand the basic meaning of the present disclosure, the summary provides a brief description of the present disclosure. The summary is not a complete description of the present disclosure and is not intended to define the technical features or scope of the claims of the present invention.

According to an implementation of the present invention, a complex for diagnosing non-alcoholic steatohepatitis is provided. The complex comprises hexa-lactoside, a metal chelator coupled to the hexa-lactoside, and a radionuclide labeling the metal chelator.

In an optional implementation of the present invention, the metal chelator is selected from a group consisting of the following substances: 1,4,7,10-tetraazacyclododecane-1,4,7,10-tetraacetic acid (DOTA), 1,4,7-triazacyclononane-1,4,7-triacetic acid (NOTA), 1,4,7-triazacyclononane-1,4-diacetic acid (NODA), and diethylenetriaminepenta-acetic acid (DTPA).

In another optional implementation of the present invention, the radionuclide is selected from a group consisting of the following substances: Ga-66, Ga-67, Ga-68, Zr-89, Lu-177, In-111, and I-123.

According to a specific implementation of the present invention, the radionuclide is Ga-68, and the metal chelator is NOTA.

According to another implementation of the present invention, a method for diagnosing non-alcoholic steatohepatitis using the complex according to any one of the foregoing implementations is provided.

In addition, according to still another implementation of the present invention, use of the complex according to any one of the foregoing implementations in preparing a pharmaceutical product for diagnosing non-alcoholic steatohepatitis is provided.

According to yet still another implementation of the present invention, a contrast agent is provided, comprising the complex according to any one of the foregoing implementations and a contrast excipient.

A person of ordinary skill in the technical field to which the present invention belongs can fully understand the central concept, adopted technical means, and various implementations of the present invention with reference to the following implementations.

### BRIEF DESCRIPTION OF THE DRAWINGS

To make the foregoing descriptions of the present invention and other objectives, features, advantages, and embodiments more apparent and understandable, the drawings are described as follows:

FIG. 1 shows results of an experiment on the function of the complex of the present invention to diagnose non-alcoholic steatohepatitis according to an implementation of the present invention.

### DETAILED DESCRIPTION

To make the description of the present disclosure more exhaustive and complete, the following provides illustrative textual descriptions for the implementations and specific embodiments of the present invention, but the implementations and specific embodiments of the present invention are not limited thereto.

Unless otherwise indicated, scientific and technical terms used in this specification have the same meaning as understood and commonly used by a person of ordinary skill in the art. Further, a noun used in this specification covers both the singular and plural forms of the noun, unless otherwise indicated.

As described in this specification, the word “about” usually means that an actual value is plus or minus 10%, 5%, 1%, or 0.5% of a specific value or range. The word “about” herein means that an actual value falls within an acceptable standard error of a mean, depending on the consideration of a person of ordinary skill in the technical field to which the present invention belongs. With the exception of examples, or unless otherwise expressly stated, it is to be understood that the ranges, quantities, values, and percentages herein are modified by the word “about”. Accordingly, unless otherwise indicated, the values or parameters disclosed in this specification and in the appended claims are all approximate values and may be changed as required.

The word “individual” refers to an animal comprising a human, particularly suitable for the complex or contrast agent of the present invention. Unless specifically noted, the word “individual” is intended to indicate both males and females.

To resolve the problem in the related art, an objective of the present invention is to provide a novel complex for diagnosing non-alcoholic steatohepatitis, comprising mainly hexa-lactoside capable of specifically binding to an asialoglycoprotein receptor (ASGPR). The expression of asialoglycoprotein receptor decreases gradually during the development of non-alcoholic steatohepatitis, and studies with immunohistological staining have proved that the expression of ASGPR decreases significantly in liver cells of animals with non-alcoholic steatohepatitis. Therefore, the expression of ASGPR can be used as a bioindicator of NAFLD. Herein, the complex of the present invention comprises hexa-lactoside highly specific to the asialoglycoprotein receptor on membranes of liver cells, and thus can be used as a pharmaceutical product for diagnosing non-alcoholic steatohepatitis. Further, the advantage of diagnosing non-alcoholic steatohepatitis using the complex of the present invention with imaging by positron emission tomography is the characteristics of non-invasiveness and high image resolution, and information about liver functions can be further obtained when steatohepatitis is diagnosed through imaging, which is also helpful for clinical medical personnel in choosing treatment, so the complex has potential for use as a molecular nuclear medicine diagnostic drug.

A plurality of examples is disclosed as follows to describe various different implementations of the present invention, so that a person of ordinary skill in the technical field to which the present invention belongs can implement the technical content disclosed in the present invention according to the disclosure of this specification. Therefore, the examples disclosed as follows may not be used to limit the scope of the claims of the present invention. Further, all references in this specification are deemed to be fully incorporated by reference into this specification.

### Example 1 Complex of the Present Invention ⁶⁸Ga-NOTA-HL

### 1.1 the Complex of the Present Invention ⁶⁸Ga-NOTA-HL has the Following Structure (Formula 1):

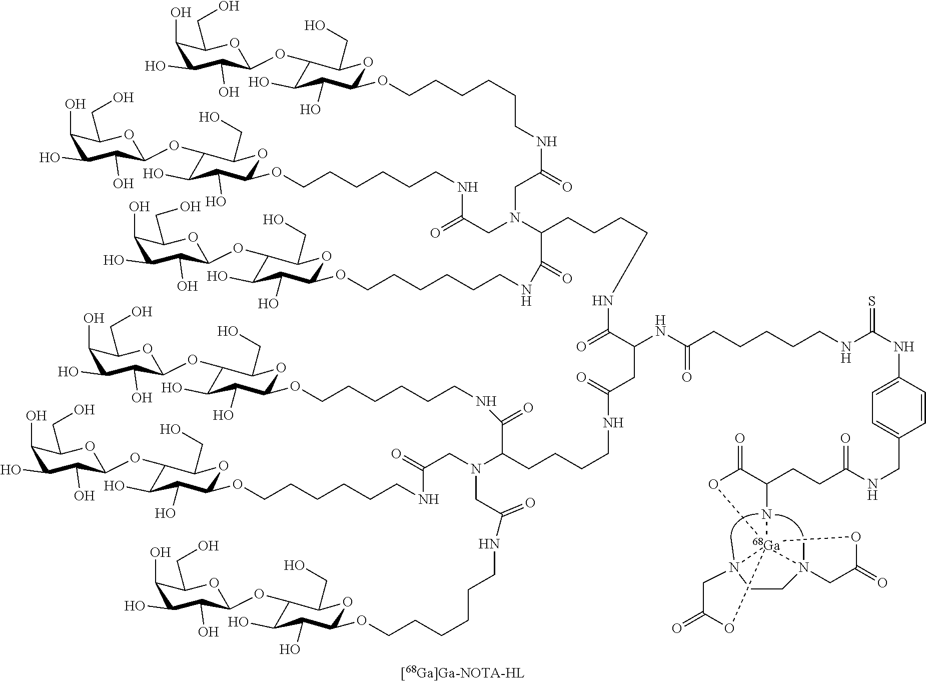

### 1.2 Preparation of the Complex of the Present Invention ⁶⁸Ga-NOTA-HL

⁶⁸Ga-NOTA-HL of the present invention may be prepared by the preparation method previously disclosed in the published literature Hung-Man Yu et al., J Label Compd Radiopharm. 2018; 61:885-894. Hexa-lactoside and p-NCS-benzyl-NOTA were added into DMF and triethylamine for reaction, subjected to extraction with ether, and then purified by RP-MPLC (with water/methanol at a flow rate of 75 mL/min), to obtain NOTA-HL. A product (with a yield of 58%) was collected and then verified by HRMS. ESI-MS was m/z 1266.6, [M+3H]³⁺ (C₁₆₁H₂₇₉N₁₇O₈₂S, molecular weight: 3797.1). NOTA-HL was placed in a small bottle and freeze-dried for use. The freeze-dried NOTA-HL in the small bottle was placed at room temperature. Then, a ⁶⁸GaCl₃ eluent from a ⁶⁸Ge/⁶⁸Ga generator was added to the freeze-dried NOTA-HL in the small bottle to react at room temperature for 15 min, to obtain ⁶⁸Ga-NOTA-HL.

### Example 2 the Complex of the Present Invention can Diagnose Non-Alcoholic Steatohepatitis

In this example, a model of an animal with non-alcoholic steatohepatitis was established using ApoE^{-/-} knockout mice. For the method for establishing the animal experimental model, refer to the published literature Robert Schierwagen et al., Seven weeks of Western diet in apolipoprotein-E-deficient mice induce metabolic syndrome and non-alcoholic steatohepatitis with liver fibrosis, Scientific Reports volume 5, Article number: 12931 (2015), https://doi.org/10.1038/srep12931.

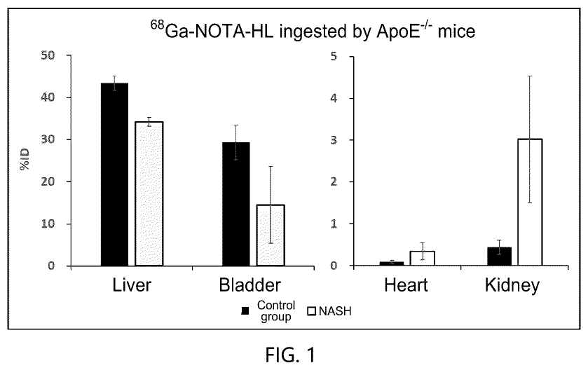

First, the ApoE^{-/-} knockout mice (each weighing about 20 g) were divided into two groups of three mice each: 1. not on a high-fat diet (control group), and 2. on a high-fat diet (NASH group), and a model of an animal with non-alcoholic steatohepatitis was established. Then, positron emission tomography was carried out with the complex of the present invention (⁶⁸Ga-NOTA-HL) prepared in Example 1 (in a dose of 300 μCi/mouse), followed by quantitative analysis for vital organs. The results are shown in FIG. 1.

In FIG. 1, at 10 minutes after injection of the complex of the present invention, the accumulation (34.21% ID) of the complex of the present invention in the liver in the NASH group is significantly lower than that in the control group (43.35% ID), the accumulation (0.34% ID) of the complex of the present invention in the heart in the NASH group is significantly higher than that in the control group (0.09% ID), the accumulation (3.02% ID) of the complex of the present invention in the kidney in the NASH group is significantly higher than that in the control group (0.44% ID), and the accumulation (14.43% ID) of the complex of the present invention in the bladder in the NASH group is significantly lower than that in the control group (29.33% ID).

Non-alcoholic steatohepatitis reduces the expression of asialoglycoprotein receptor on membranes of liver cells. As the complex of the present invention can specifically bind to the asialoglycoprotein receptor, and the foregoing data shows that the accumulation of the complex of the present invention in the liver in the NASH group is significantly reduced compared with the control group, it can be proved that the complex containing hexa-lactoside of the present invention can be used to diagnose non-alcoholic steatohepatitis.

In conclusion, the present invention provides a complex for diagnosing non-alcoholic steatohepatitis. The complex of the present invention can specifically bind to an asialoglycoprotein receptor, a bioindicator of non-alcoholic steatohepatitis, with good sensitivity, so that the complex can be used as a radiological diagnostic agent for diagnosing non-alcoholic steatohepatitis.

What is claimed is:

1. A complex for diagnosing non-alcoholic steatohepatitis, comprising:
hexa-lactoside;
a metal chelator, coupled to the hexa-lactoside; and
a radionuclide, labeling the metal chelator.

2. The complex according to claim 1, wherein the metal chelator is selected from a group consisting of the following substances: 1,4,7,10-tetraazacyclododecane-1,4,7,10-tetraacetic acid (DOTA), 1,4,7-triazacyclononane-1,4,7-triacetic acid (NOTA), 1,4,7-triazacyclononane-1,4-diacetic acid (NODA), and diethylenetriaminepenta-acetic acid (DTPA).

3. The complex according to claim 2, wherein the radionuclide is selected from a group consisting of the following substances: Ga-66, Ga-67, Ga-68, Zr-89, Lu-177, In-111, and I-123.

4. The complex according to claim 3, wherein the radionuclide is Ga-68.

5. The complex according to claim 4, wherein the metal chelator is NOTA.

6. Use of the complex according to claim 1 in preparing a pharmaceutical product for diagnosing non-alcoholic steatohepatitis.

7. The use according to claim 6, wherein the radionuclide is Ga-68.

8. The use according to claim 7, wherein the metal chelator is NOTA.

9. A contrast agent, comprising:
the complex according to claim 1; and
a contrast excipient.

10. The contrast agent according to claim 9, wherein the radionuclide is Ga-68, and the metal chelator is NOTA.
